(19) United States
(12) Patent Application Publication
(10) Pub. No.: US 2012/0114763 A1
(43) Pub. Date: May 10, 2012

(54) Method for Producing Collagen/Apatite Composite Membrane for Guided Bone Regeneration

(75) Inventors: Hyoun-Ee Kim, Seoul (KR); Hae-Won Kim, Gwacheon (KR); Ju-Ha Song, Seongnam (KR); Sung-Min Chung, Seoul (KR)

(73) Assignees: GENOSS LTD, Suwon-Gyeonggi-do (KR); SEOUL NATIONAL UNIVERSITY INDUSTRY FOUNDATION, Seoul (KR)

(21) Appl. No.: 12/093,591

(22) PCT Filed: Nov. 14, 2005
(86) PCT No.: PCT/KR05/03855

§ 371 Date: Sep. 19, 2008

(51) Int. Cl. A61K 33/42; A61P 19/00

(52) U.S. Cl. 424/602

(57) ABSTRACT

Disclosed is a method for producing a collagen/apatite composite membrane useful as a barrier to protect fibrous connective tissues from infiltrating into a site of defective tissue or bone in guided tissue regeneration (GTR) or guided bone regeneration (GBR) procedures. Such composite membrane can be obtained by the steps of co-precipitating collagen and apatite in aqueous basic solution in a very well controlled manner, followed by casting the precipitation onto a filter paper, lyophilization and cross-linking. The method according to the present invention can allow forming a collagen/apatite composite membrane in which the crystals of apatite, in particular, hydroxyapatite are coated on and along with the collagen fiber direction without aggregation of self-assembled collagen fibers. Such composite membrane has excellent biological and mechanical properties such as biocompatibility, cell affinity, biodegradability and form stability, and therefore it can be used as a barrier for GTR. The method of the present invention can also allow the composite membrane to have microstructure enabling osteoblasts to be attached and proliferated well on the membrane, and can allow the properties of membrane to be controlled by adjusting its thickness in the formation of membrane, thereby providing a collagen/apatite composite membrane suitable to a needed use.

### TECHNICAL FIELD

The present invention relates to methods for producing collagen/apatite composite membranes by biomimetic processes usefully applicable for guided bone or tissue regeneration and collagen/apatite composition membranes produced by the same methods.

### BACKGROUND ART

Recently, tissue engineering has become the best solution for reconstruction of defective tissues in both medical and engineering fields on the basis of the developments up to now. One of the best developed fields is guided bone regeneration (GBR) or guided tissue regeneration (GTR). This is to use a barrier in a form of membrane to protect fibrous connective tissue from infiltrating into a site of defective bone or tissue.

Many researchers have tried to make materials very well suitable to the membranes for such use, but all the materials as tried up to now have advantages and shortcomings, respectively. For example, polytetrafluoroethylene (PTFE) membranes have been used as such barriers for GTR, but the PTFE membranes have the fatal shortcoming that they should be removed with secondary operation due to their bioinertness and they also have limitations of defective tissue sizes applicable to reconstruct new tissues.

In order to overcome these shortcomings, many researchers have suggested and studied resorbable membranes such as those of collagen and synthetic biodegradable polymers. Collagen, which is an inert natural protein found in human and animal skins, connective tissues, bones and teeth, has excellent biocompatibility and resorbability, but has poor mechanical properties. In addition, the solubility of pure collagen is generally too high. On the other hand, polylactide-based polymers have sufficient mechanical properties, but their cell affinities and dissolution rates are too low [Y. Aman, M. Ota, K. Sekiguchi, Y. Shibukawa, and S. Yamada, Evaluation of a poly-1-lactic acid membrane and membrane fixing pin for guided tissue regeneration on bone defects in dogs, Oral Surg Oral Med Oral Pathol Oral Radiol Endod 2004; 97: 155-163; M. Asboe, E. M. Pinholt and E. Hjorting-Hansen, Healing of experimentally created defects: a review, British Journal of Oral and Maxillofacial Surgery 1995; 33: 312-318; Y. Park, Y. Ku, C. Chung and S. Lee, Controlled release of platelet-derived growth factor from porous poly(L-lactide) membranes for guided tissue regeneration, Journal of Controlled Release 1998; 51: 201-211; M. Kikuchi, Y. Koyama, T. Yamada, Y. Imamura, T. Okada, N. Shirahama, K. Akita, K. Takakuda and J. Tanaka, Development of guided bone regeneration membrane composed of β-ticalcium phosphate and poly(L-lactide-co-glycolide-co-ε-caprolactone) composites, Biomaterials 2004; 25: 5979-5986].

Considering these problems, some researchers have prepared composites of osteoconductive calcium phosphate ceramics and biodegradable synthetic polymers. For example, hydroxyapatite/poly(ε-caprolactone) composites [D. Choi, K. G. Marra and P. N. Kumta, Chemical synthesis of hydroxyapatite/poly(ε-caprolactone) composites, Materials Research Bulletin 2004; 39: 417-432], biodegradable Polyactive®/hydroxyapatite membrane [J. A. Jansen, J. E. de Ruijter, P. T. M. Janssen and Y. G. C. J. Paquay, Histological evaluation of biodegradable Polyactive®/hydroxyapatite membrane, Biomaterials 1995; 16: 819-827], β-ticalcium phosphate and poly(L-lactide-co-glycolide-co-ε-caprolactone) composites [M. Kikuchi, Y. Koyama, T. Yamada, Y. Imamura, T. Okada, N. Shirahama, K. Akita, K. Takakuda and J. Tanaka, Development of guided bone regeneration membrane composed of βticalcium phosphate and poly(L-lactide-co-glycolide-co-ε-caprolactone) composites, Biomaterials 2004; 25: 5979-5986] have been investigated. Although their feasibility for GBR has increased, they have a problem in the uniformity of the ceramic particles incorporated in the polymer matrix according to the particle size or amounts of particles. Moreover, the ceramic particles sometimes act as a defect factor and decrease the mechanical properties of composite membranes.

Another approach to composite membranes for GTR is to form hydroxyapatite in and on collagen membranes. To enhance the forming ability of membrane, many methods of collagen modification have been suggested [S. Rhee and J. Tanaka, Effect of citric acid on the nucleation of hydroxyapatite in a simulated body fluid, Biomaterials 1999; 20: 2155-2160; D. Lickorish, J. A. M. Ramshaw, J. A. Werkmeister, V. Glattauer and C. R. Howlett, Collagen-hydroxyapatite composite prepared by biomimetic process, J Biomed Mater Res 68A 2004; 19-27; A. Tampieri, G. Celotti, E. Landi, M. Sandri, N. Roveri and G. Falini, Biologically inspired synthesis of bone-like composite: Self-assembled collagen fibers/hydroxyapatite nanocrystals, J Biomed Mater Res 67A 2003; 618-625]. More recently, collagen/hydroxyapatite composites were made by the biomimetic approach. In this method, apatite crystals were precipitated and aligned along with collagen fiber direction. The advantage of this method is to make a nanoscale-ordered and homogeneous structure of composites. However, this process posed serious problems because self-assembled fibers were aggregated and became irregular particles [M. Kikuchi, S. Itoh, S. Ichinose, K. Shinomiya and J. Tanaka, Self-organization mechanism in a bone-like hydroxyapatite/collagen nanocomposite synthezed in vitro and its biological reaction in vivo, Biomaterials 2001: 22: 1705-1711; S. Itoh, M. Kikuchi, Y. Koyama, K. Takakuda, K. Shinomiya and J. Tanaka, Development of an artificial vertebral body using a novel biomaterial, hydroyapatite/collagen composite, Biomaterials 2002; 23: 3919-3926].

U.S. Pat. Nos. 6,300,315 and 6,417,166 disclosed a method for obtaining a sheet of mineralized collagen membrane by simultaneously adding calcium chloride solution and ammonium phosphate solution into collagen gel slurry and casting the precipitation on a filter paper. This method is characterized to use collagen slurry as starting material of collagen to precipitate apatite precipitation on the collagen and obtain the collagen membrane. Meanwhile, the method, in which collagen slurry is used to form apatite precipitation thereon, had already been disclosed by the previous U.S. Pat. No. 5,320,844 to the same inventor (which was registered and published before the filing dates of the above later patents), whereby this patent only suggested the composite as prepared by the method might be used as an alternative of light tissue, but suggest no membrane for GTR and preparing method therefor. This inventor described in the later patents that mineralized collagen had heretofore (up to the filing date) been prepared by freeze-drying to form a sponge structure or was alternatively prepared in solid block or granule form, however, none of these mineralized forms of collagen could be used as membrane barriers for GTR procedures and no collagen membrane suitable for GTR application suggested.

In the meantime, researches to alternate defective bones to artificial bone materials have considerably proceeded. Korean Patent Publication No. 2005-0083797 (which was published on Aug. 26, 2005, and was in Korean national phase of international publication No. WO 2004/041320 on May 21, 2004) disclosed cross-linked porous apatite/collagen composite useful as artificial bone material and manufacturing method therefor. This patent application was finally to obtain artificial bone material by the steps of mixing aqueous phosphate solution of collagen and dispersion of calcium hydroxide to obtain 8/2 by weight of apatite/collagen composite, preparing dispersion in a form of paste from such composite, gelatinizing such paste to form a molding and lyophilizing and cross-linking such molding. This patent application did not mention any membrane for GTR, but only suggested bone-alternative material in which apatite as main component compounded with collagen.

### DISCLOSURE OF INVENTION

### Technical Problem

The object of the present invention is to provide a method for producing a collagen/apatite composite membrane useful as a barrier to protect fibrous connective tissues from infiltrating into a site of defective tissue or bone in guided tissue regeneration (GTR) or guided bone regeneration (GBR) procedures.

The object of the present invention is also to provide a collagen/apatite composite membrane produced by the same method as mentioned above.

### Technical Solution

The present invention provides a method for producing a membrane comprising collagen/apatite composite in which the apatite is uniformly precipitated on the collagen by biomimetic approach.

The method of the present invention comprises the steps of preparing aqueous solution containing a soluble calcium ion (calcium ion solution), preparing phosphate/collagen solution in which collagen is dissolved into aqueous solution (phosphate solution) containing soluble phosphate ion, preparing buffer solution adjusted with ph 7 or higher, adding the calcium ion solution and the phosphate/collagen solution into the buffer solution and stirring the mixture to form precipitation of collagen/apatite composite, and washing and filtering such precipitation of collagen/apatite composite on a filter paper and then lyophilizing the precipitation to obtain a collagen/apatite composite membrane.

The method of the present invention may further include the step of cross-linking the collagen of said collagen/apatite composite membrane. Such cross-linking of collagen may be performed by immersing said collagen/apatite composite membrane into EDC-NHS solution in alcohol in which 1-ethyl-3-(3-dimethyl aminopropyl)carbodiimide (EDC) and N-hydroxysuccinimide (NHS) is dissolved in alcohol. The alcohol used as a solvent in said EDC-NHS solution is preferably ethanol. The method of the present invention may also further include the steps of, after said cross-linking, recovering and washing said cross-linked membrane with pure water and then lyophilizing it. The method may also further include the step of adjusting the thickness of said membrane by drying said membrane in the condition of uniformly pressing the surface of said membrane at a wet atmosphere.

In the method of the present invention, said calcium ion solution and said phosphate/collagen solution is preferably prepared to allow the nominal component ratio of collagen and apatite, which could be obtained in the case if the reaction to form said precipitation of collagen/apatite composite were perfectly performed without any loss, to be a value in the range of 8:2˜6:4 by weight. Said calcium ion solution preferably is solution in which Ca(OH)₂ is dissolved into water, and said phosphate/collagen solution preferably is solution in which collagen is dissolved into aqueous H₃PO₄ solution. Said step of forming the precipitation of said collagen/apatite composite is preferably performed on the condition that the mixture is kept at pH 7 or higher, and more preferably, at pH 9. Said buffer solution is preferably, tris-HCl buffer solution established at pH 9 and said step of forming the precipitation of said collagen/apatite composite is preferably performed on the condition that the mixture is kept at pH 9. Said steps of washing and filtering the precipitation of said collagen/apatite composite is preferably performed by the steps of separating such precipitation by means for separating solid and liquid, washing the precipitation and then casting the precipitation onto a filter paper, or said steps of washing and filtering is preferably performed by the steps of casting such precipitation onto a filter paper and then washing it on the filter paper. The apatite in said collagen/apatite composite is preferably, hydroxyapatite.

### Advantageous Effects

The method according to the present invention can allow to form a collagen/apatite composite membrane in which the crystals of apatite, in particular, hydroxyapatite are coated on and along with the collagen fiber direction without aggregation of self-assembled collagen fibers. Such composite membrane has excellent biological and mechanical properties such as biocompatibility, cell affinity, biodegradability and form stability, and therefore it can be used as a barrier for GTR. The method of the present invention can also allow the composite membrane to have microstructure enabling osteoblasts to be attached and proliferated well on the membrane, and can allow the properties of membrane to be controlled by adjusting its thickness in the formation of membrane, thereby providing a collagen/apatite composite membrane suitable to a needed use.

### BRIEF DESCRIPTION OF THE DRAWINGS

FIG. 1(A) is a schematic diagram explaining processes in the method of the present invention and FIG. 1(B) is photographs showing collagen/hydroxyapatite composite membranes before cross-linking produced by the method of the present invention.

FIG. 2 is graphs showing analyses of collagen/hydroxyapatite composite membranes produced by the examples according to the present invention, wherein (A) is a graph of Thermogravimetric Analysis (TGA), and (B) is a graph of FT-IR spectroscopic analysis. At each graph, (a) is for a cross-linked pure collagen membrane, (b) is for a cross-linked collagen/hydroxyapatite composite membrane with 20% by weight (nominal component ratio) of hydroxyapatite, and (c) is for a cross-linked collagen/hydroxyapatite composite membrane with 40% by weight (nominal component ratio) of hydroxyapatite.

FIG. 3 is microscopic images showing morphologies of the collagen/hydroxyapatite composite membranes produced by the examples according to the present invention, wherein (A) is an SEM image showing a surface of a cross-linked pure collagen membrane, and as SEM images of the cross-linked collagen/hydroxyapatite composite membrane with 40% by weight (nominal component ratio) of hydroxyapatite, (B) is for a surface, (C) is for a cross-section, and (D) is for exposed microfibers, and (E) shows a TEM image and SAD pattern.

FIG. 4 is a graph showing density distributions of the cross-linked collagen/hydroxyapatite composite membranes produced by the examples according to the present invention with thicknesses, wherein all the specimens have same areas by filtering with same means and method.

FIG. 5 is a graph showing the mechanical properties of the cross-linked collagen/hydroxyapatite composite membranes produced by the examples according to the present invention, wherein (A) is a curve of stress-strain, and (B) shows tensile strength and elastic modulus (n>6, *p<0.01).

FIG. 6 shows form stabilities of the cross-linked collagen/hydroxyapatite composite membranes produced by the examples according to the present invention, wherein (A) is a photograph showing the level of swelling in water and (B) is a graph showing the change of volume after the membranes according to the present invention were immersed into water for 24 hours (*p<0.05).

FIG. 7 shows biodegradable stabilities of the cross-linked collagen/hydroxyapatite composite membranes producing the examples according to the present invention, wherein (A) is a photograph showing the status after the membranes according to the present invention were treated with 150 U/ml of collagenase solution for 24 hours, and (B) is a graph showing the level of collagen biodegradability. All the membranes used in (B) were cross-linked by EDC/NHS and cultivated with 150 U/ml of collagenase at 37° C. for 24 hours. The values are average±standard deviation (*p<0.05, p<0.01).

FIG. 8 is a schematic diagram showing a mechanism of collagen biodegradation by collagenase.

FIG. 9 shows cell characteristics of the cross-linked collagen/hydroxyapatite composite membranes produced by the examples according to the present invention, wherein (A) is a graph showing the characteristics of cell growth and (B) is photographs of SEM images, 3 days later after MT3T3 osteoblasts were cultivated on the membranes. In the (B), (a) and (b) are for pure collagen membrane, and (c) and (d) are for the membrane with 80% by weight (nominal component ratio) of collagen. The amount of cells were not largely different. Granulates were formed on the composite membrane and near the cells on the composite membrane.

### BEST MODE FOR CARRYING OUT THE INVENTION

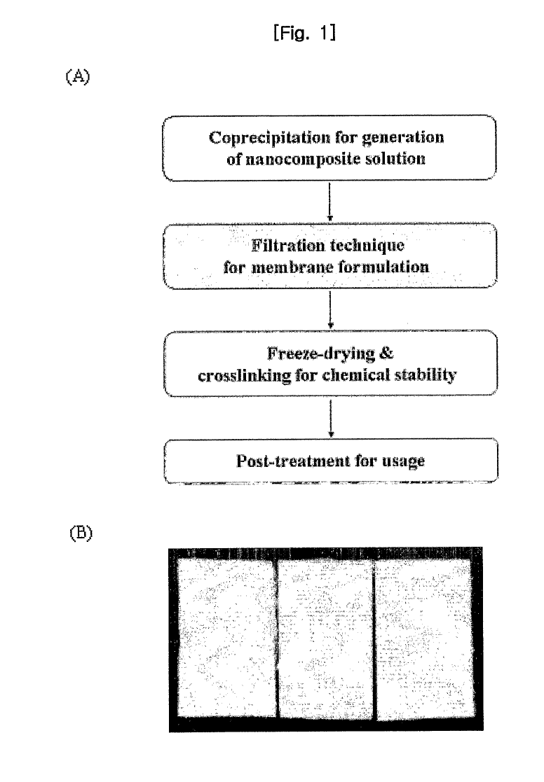

The present invention relates to a method for producing a collagen/apatite composite membrane by biomimetic approach. FIG. 1(A) is a schematic diagram explaining processes in the method of the present invention and FIG. 1(B) is photographs showing collagen/hydroxyapatite composite membranes before cross-linking produced by the method of the present invention.

In order to produce a collagen/apatite composite membrane in accordance with the method of the present invention, soluble calcium ion containing aqueous solution (calcium ion solution) is prepared. The calcium compound contained in the calcium ion solution is not particularly restricted if it is soluble into water and does not prohibit to precipitate apatite, and the examples in accordance with the present invention used calcium hydroxide [Ca(OH)₂], in particular. Such calcium compound is dissolved into water with a needed amount at a concentration within the limit of solubility. The needed amount of calcium compound is determined in consideration to the needed component ratio of the collagen and apatite, which will be formed through precipitation, in the final membrane.

Phosphate/collagen solution in which collagen is dissolved into soluble phosphate ion containing aqueous solution (phosphate solution) is also prepared. The order for dissolutions of phosphoric acid or phosphate and collagen is not particularly restricted. The amounts of collagen and phosphate are determined in consideration to the needed component ratio of the collagen and apatite in the final membrane. The phosphate compound contained in the phosphate solution is not particularly restricted if it is soluble into water and does not prohibit to precipitate apatite, and the examples in accordance with the present invention used phosphoric acid (H₃PO₄), in particular. Such phosphate compound is dissolved into water with a needed amount at a concentration within the limit of solubility. The collagen used in the present invention is not particularly restricted, but preferably, is type I collagen. The concentration of collagen may be appropriately determined within the limit of solubility.

Meanwhile, as separate to these reactants, buffer solution adjusted to pH 7 or higher is prepared. The buffer solution is not particularly restricted if it does not prohibit precipitation of collagen and apatite and formation of apatite, but tris-HCl buffer solution was preferably used in the examples in accordance with the present invention. The pH of the buffer solution is more preferably established to about pH 9. If a reaction for precipitation of apatite is performed at about pH 9, then hydroxyapatite may be formed.

Into the prepared buffer solution are added the calcium solution and phosphate/collagen solution simultaneously or in order. The mixture of the reactants is vigorously stirred to induce co-precipitation of apatite and collagen. The pH of the reactant mixture is preferably kept at pH 7 or higher, and more preferably, at pH 9. The adjustment and maintenance of pH of the reactant mixture is achieved by using buffer solution, acid such as HCl and base such as NH₄OH. The collagen/apatite composite formed by such co-precipitation has a microstructure in which apatite, especially hydroxyapatite is uniformly coated on collagen fiber in a form that C axis of hydroxyapatite is aligned along with the collagen fiber direction. Important consideration in the inducement of such co-precipitation is uniform precipitation and is to prevent aggregation of collagen by self-assemblage. These things are particularly important matters if the component ratio of collagen is high in the collagen/apatite composite. The nominal component ratio of collagen/apatite is 9/1˜5/5 by weight, preferably 8/2˜6/4 by weight in the composite membrane of the present invention. In this specification, the nominal component ratio of collagen/apatite does not means the actual ratio in the final collagen/apatite composite membrane, but means the ratio of collagen/apatite on the ideal condition that the co-precipitation reaction of the reactants for the co-precipitation of collagen and apatite proceeds perfectly with no loss.

If the composite membrane requires a high ratio of collagen, then the concentration of collagen in the reactant mixture become high to increase the possibility of collagen to be aggregated by its self-assemblage. In order to prevent this problem, collagen slurry may be used, but in this case, the uniform precipitation of apatite on collagen is limitedly achieved. In addition to the difficulty for the perfect uniformity of apatite precipitation due to the limitation to the uniformity of collagen slurry, the precipitation of apatite cannot be formed on the parts of collagen slurry not exposed, thereby it being difficult to obtain the composite membrane with the microstructure of uniform apatite precipitation on collagen. On these reasons, collagen is preferred to be dissolved into water in the present invention.

On the other hand, in the event that the co-precipitation of collagen and apatite is induced from dispersion of calcium salt with the limited solubility, there may occur non-uniformity of apatite precipitation distribution on collagen together with non-uniformity of the precipitated apatite composition. Therefore, the solution of the calcium salt with the limited solubility, particularly, calcium hydroxide, not the dispersion, is used in the present invention.

In the co-precipitation reaction system used in the present invention, the amount of collagen is larger than that of apatite, in particular, hydroxyapatite and preferably, the component ratio of collagen/apatite is in the range of 9/1˜5/5, and more preferably, in the range of 8/2˜6/4. The possibility of the little amount of apatite to contact or meet with the collagen is low in such reaction system, and therefore, nano-scale size of apatite particulates may be precipitated on the collagen uniformly and in a special orientation and the aggregation of the self-assembled collagen may be prevented in the solution. Such reaction system is consisted of the combination of calcium ion solution, collagen solution, buffer solution and pH adjustment and maintenance, and may be reinforced with vigorous stirring, low concentration, and long stirring and aging for 12 hours or more, preferably, for 24 hours or more. It is preferable that the reaction temperature should be about 40° C., and more precisely 37° C.

In the next, the precipitation of collagen/apatite composite formed by the biomimetic co-precipitation reaction system is washed, filtered and lyophilized to form collagen/apatite composite membrane without cross-linkage. At this time, the filtering is to cast the composite precipitation onto filter paper to form a sheet of membrane. The washing may be to wash the composite precipitation with pure water after casting it on filter paper, or may be to separate the precipitation by a solid-liquid separation means, for example, centrifugation or filtering and then to disperse it into water again and cast it onto filter paper. The lyophilization may be performed by any known method. It is generally known that the more the speed of lyophilization is, the smaller in tendency the size of pore in the porous body is and so, the pore size and form of the porous body obtained by lyophilization may be controlled with the lyophilization speed. Therefore, the present invention may also produce a composite membrane with suitable pore size for a certain use.

It is preferable to cross-link the prepared collagen/apatite composite membrane for enhancing form stability, namely, for providing stable form maintenance ability to resist against swelling in water when it is used in a body as well as for enhancing mechanical properties. The cross-linkage of composition membrane may be performed in a physical or chemical way, and may simply be performed by immersing the lyophilized composite membrane into a solution of cross-linking agent. The cross-linking agent may include agents in types of aldehydes, isocyanates, carbodiimides and others. The examples of the present invention used EDC-NHS solution in which 1-ethyl-3-(3-dimethyl aminopropyl)carbodiimide (EDC) and N-hydroxysuccinimide (NHS) are dissolved into ethanol. The use of alcohol such as ethanol as solvent may allow preventing the dissolution or loss of the composite membrane in the cross-linking reaction. After the cross-linking reaction, the composite membrane is washed to remove the remaining cross-linking agent and lyophilized again.

Meanwhile, the collagen/apatite composite membranes as prepared by the above method of the present invention are sheets of membrane formed by casting the precipitation of collagen/apatite composite from the co-precipitation reaction onto the filter papers with same area, but there is limitation on the uniformity of thickness although the thickness may be uniformly adjusted by the precipitation and filtration. Therefore, the present invention can dry the composite membrane, for example, on the condition or after the surface of membrane is uniformly pressed under wet atmosphere at 40° C. to obtain the composite membrane with uniform and needed thickness. At this time, the density and porosity of the composite membrane can be adjusted by the adjustment of thickness in the present invention, and therefore, the present invention can provide the composite membrane with the adjusted thickness suitable for a certain needed use.

### Mode for the Invention

Now, examples and further explanations of the present invention will be offered for the purpose of illustration, not for limitation of the scope of the present invention.

### Examples

### 1. Preparation of Cross-Linked Collagen/Hydroxyapatite Composite Membrane

(1) Preparation of Collagen/Hydroxyapatite Composite Membrane

As starting materials, Ca(OH)₂ (99.995%, Aldrich, USA), H₃PO₄ (99.999%, 85 wt % aqueous solution, Aldrich, USA), and Type I collagen (MW 300,000, RegenMed. Inc., Korea) were used. The precipitation method was modified from the biomimetic method by co-precipitation [M. Kikuchi, S. Itoh, S. Ichinose, K. Shinomiya and J. Tanaka, Self-organization mechanism in a bone-like hydroxyapatite/collagen nanocomposite synthesized in vitro and its biological reaction in vivo, Biomaterials 2001: 22: 1705-1711; B Y Yoon, H W Kim, S H Lee, C J Bae, Y H Koh, Y M Kong and H E Kim, Stability and cellular responses to fluorapatite-collagen composites, Biomaterials 2005; 26: 2957-2963]. Ca(OH)₂ was dissolved in cold distilled water completely in the range of solubility. Separately, collagen was dissolved in the H₃PO₄ solution of which 59.7 mM was diluted in distilled water. Before the reaction, the tris-HCl buffer solution with pH 9 was prepared keeping the temperature at 37° C. by water bath. Both Ca(OH)₂ and H₃PO₄/collagen solution was added at the same time into a reaction vessel containing the buffer solution. During the reaction, care was taken to maintain the pH at 9 using HCl and NH₄OH. The amount of Ca, P and collagen were set to produce the final collagen/HA ratio of 80/20 and 60/40 (wt/wt). The mixture was stirred fast during 24 hrs at 37° C. in water bath. The precipitates were subsequently filtrated and washed over again until the volume was 50 ml and freeze-dried at −60° C. under vacuum.

As a reference, a pure collagen membrane was made by the same filtering method. Collagen was dissolved in 50 mM acetic acid solution. After the collagen solution was mixed with tris-HCl buffer until the pH to 9, the solution was aged at 37° C. during 24 hrs. The precipitated collagen fibers were filtrated, washed over again and lyophilized at −60° C. under vacuum.

(2) Cross-Linkage

Chemical cross-linking of collagen membranes was performed using 1-ethyl-3-(3-dimethyl aminopropyl)carbodiimide (EDC) and N-hydroxysuccinimide (NHS) [L. H. H. Olde Damink, P. J. Dijkstra, M. J. A. l van Luyn, P. B. van Wachem, P. Nieeuwenhuis and J. Feijen, Cross-linking of dermal sheep collagen using a water-soluble carbodiimide, Biomaterials 1996; 17: 765-773]. The membranes were immersed in 100 mM EDC 100 mM NHS solution in 95% ethanol for 24 hrs at room temperature. As a solvent of EDC and NHS, ethanol prevented the dissolution or loss of the membrane during the process [M. Chang and J. Tanaka, FT-IR study for hydroxyapatite/collagen nanocomposite cross-linked by glutaraldehyde, Biomaterials 2002; 23: 4811-4818]. The cross-linked membranes were carefully withdrawn and washed with sufficient distilled water for 5 min five times to remove the residual EDC and NHS. Afterward washing, the membranes were re-freezed and lyophilized at −60° C. under vacuum.

### 2. Characterization of Cross-Linked Collagen/Hydroxyapatite Composite Membrane

(1) Identification of Chemical Composite and Microstructure

The thermogravimetric analysis (TGA) was performed to determine the amount of hydroxyapatite on composites. Each membrane 20 mg was studied using a TG analyzer (TGA-1000, Rheometric Scientific, UK) and measurements were recorded from 30° C. to 900° C. with 10° C./min heating rate under air. The remnants were recognized as inorganic components, mainly hydroxyapatite. The phase of the composition was analyzed by X-ray diffraction (XRD, M18XHF-SRA, MAC Science, Yokohama, Japan) patterns with CuKα radiation within 25-35° (2θ) at a rate of 1°/min. Chemical analysis of the composites was conducted by a Fourier transform infrared (FT-IR) spectrometer (Nicolet Magma 550 series II, Midac, USA) over the range between 4000 and 400 cm⁻¹ at 1 cm⁻¹ resolution averaging 64 scans. From the analysis, cross-linking of membrane was confirmed [M. Chang and J. Tanaka, FT-IR study for hydroxyapatite/collagen nanocomposite cross-linked by glutaraldehyde, Biomaterials 2002; 23: 4811-4818]. The microstructure and nano-structure of the composites were observed by field-emission scanning electron microscopy (FE-SEM, JSM-6330F, JEOL, Tokyo, Japan) and transmission electron microscopy (TEM, CM-20, Philips Electron Optics, Netherlands).

(2) Mechanical Properties

Density of each type of specimens was measured and compared with theoretical density. The membranes, which had a wide range of thickness from 200 to 1000□, were cut into a square shape with dimensions 5×5 mm and weighed. Tensile strength and elastic modulus were assessed using a universal testing machine (Model 5565, Instron Corp., Danvers, Mass.). The membranes were warm-pressed to have uniform thickness of 500□ under wet condition and lyophilized at −6° C. under vacuum. The exact thickness of dried specimens was measured using a confocal laser scanning microscope (CLSM, OLS 1200, Olympus). Then the membranes were cut into a dumbbell shape with a length of 25 mm and inner width of 5 mm. A tensile force was applied at an extension rate of 1 mm/min. More than six specimens were tested for each condition.

(3) Degree of Swelling

The swelling degree of membranes was studied by the volume change before and after soaking. The volume of cross-linked collagen and collagen/HAp membranes was measured and then they were separately immersed in distilled water at room temperature for 24 hrs. After removal from water, they were laid on the moist tissue for 1 min until no dripping water was observed. The volume of the swollen membranes was measured and the degree of volume change was calculated by the following equation: volume change (%)=[(VₛV_{d})/V_{d}]×100 where V_{d} is the volume of the dry membrane and Vₛ is the volume of the swollen membrane.

(4) In Vitro Cellular Assay

MC3T3-E1 preosteoblasts (ATCC, CRL-2593) were cultured in regular culture media consisting of α-modified minimum essential medium (α-MEM; Join Bio Innovation (JBI), Korea) supplemented with 10% heated-inactivated fatal bovine serum (FBS; GIBCO, USA) and 1% antibiotic/antimycotic (GIBCO, USA) in a humidified atmosphere of 5% CO₂ at 37° C. Before the experiments, MC3T3 cells were trypsinized and plated on the specimens at a density of 1×10⁵ cells/cm² and cultured osteogenic media (regular culture media described above plus 10 mM β-glycerol phosphate and 50□/ml L-ascorbic acid (Sigma, USA)) before they were harvested at 1 and 3 days for cell morphology. The cell morphology was observed with SEM after fixing with glutaraldehyde (2.5%), dehydrating with graded ethanols (75, 90, 95 and 100%), critical point drying and gold coating.

(5) In Vitro Collagenase Degradation

The biological stability of the cross-linked collagen and composite membranes was evaluated by in vitro collagenase biodegradation test [L. Ma, C. Gao, Z. Mao, J. Zhou and J. Shen, Enhanced biological stability of collagen porous scaffolds by using amino acids as novel cross-linking bridges, Biomaterials 2004; 25: 2997-3004]. Each kind of membranes was incubated in phosphate buffered saline (PBS, pH 7.4) containing given concentrations of type I collagenase (273 U/mg, Sigma) at 37° C. during 24 hrs. The degradation was stopped by incubating the assay in an ice bath immediately. Following centrifugation at 1500 rpm for 10 min, the clear supernatant was hydrolyzed with 6M HCl at 110° C. for 24 hrs. The content of hydroxyproline released from the collagen molecules in the membranes was measured using ELISA method [W. D. Coasts, Jr, D. T. Cheung, B. Han, J. W Currier and D. P. Faxon, Ballon angioplsty significantly increases collagen content but does not alter collagen subtype I/III ratios in the atherosclerotic rabbit iliac model, J Mol Cell Cardiol 1996; 28: 441-446]. The biodegradation degree is defined as the percentage of the released hydroxyproline from the membranes to the completely degraded one with the same composition and weight. Also, the amount of degradable collagen in the solution was measured to find out the effect of HAp in the degradation process.

(6) Statistical Analysis

Data are expressed as the mean±standard deviation (SD). Statistical analysis was performed using the two-population Student's t-test. The significant level was set as p<0.05.

### 3. Results and Discussions

(1) Identification of the Compositions of Membranes

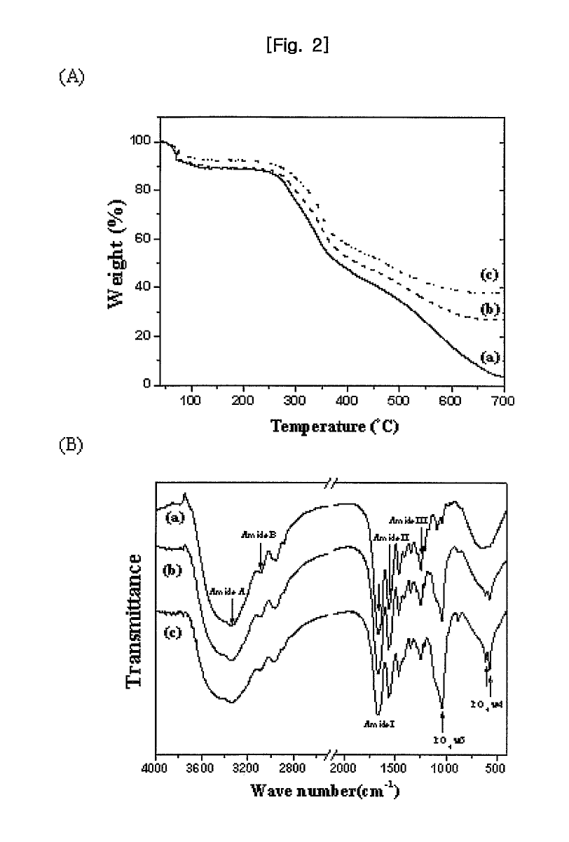

FIG. 2 is graphs showing analyses of collagen/hydroxyapatite composite membranes produced by the examples according to the present invention, wherein (A) is a graph of Thermogravimetric Analysis (TGA), and (B) is a graph of FT-IR spectroscopic analysis. At each graph, (a) is for a cross-linked pure collagen membrane, (b) is for a cross-linked collagen/hydroxyapatite composite membrane with 20% by weight (nominal component ratio) of hydroxyapatite, and (c) is for a cross-linked collagen/hydroxyapatite composite membrane with 40% by weight (nominal component ratio) of hydroxyapatite.

The amounts of HA in the collagen/hydroxyapatite (Col/HA) membranes were identified by TGA analyses as shown in FIG. 2. The amounts of the reactants in the co-precipitation reaction for preparing the composite membrane was used to allow the nominal composition ratio of the product Col/HA composite membrane to be 80/20 and 60/40 by weight, but it was identified that the actual ratio was 82/18 and 65/35 be weight. Such difference between the nominal and actual composition ratios was attributable to the incomplete reactions of the formation/precipitation of hydroxyapatite. There was no significant difference among the TG curves of pure collagen and Col/HA composites in terms of collagen decomposition behavior: the first weight loss of 5˜10% occurring at 50˜1000° C. water in the specimens; and the second weight loss at 240˜250° C.

The yields of the formation/precipitation of HA could be calculated from the compositions of the final composite membranes calculated from the results of TGA. The nominal composition of 80/20 of Col/HA was considerably deviated from the actual composition of the composite membrane, which assumed to be resulted from the aggregation of self-assembled collagen fibers. It was difficult to find nano-scale fibers in the composite membrane with this composition by observation of the SEM morphology. Therefore, it is preferable that the composition of Col/HA in the present invention should be 80/20 or less.

As shown in FIG. 2(B), typical IR bands for collagen are observed; N—H stretching at ˜3310 cm⁻¹ for the amide A, C—H stretching at ˜3063 cm⁻¹ for the amide B, C═O stretching at 1600-1700 cm⁻¹ for the amide I, N—H deformation at 1500-1550 cm⁻¹ for the amide II and N—H deformation at 1200-1300 cm⁻¹ for the amide III band. It was reported that the spectral feature of the amide B band arising from C—H stretching was considerably influenced by the cross-linking. It could be identified from this fact that the composite membranes of the present invention were cross-linked. Bands in regard with HA include OH stretching (3569 cm⁻¹) and phosphate bands. FIG. 2(B) also shows the phosphate bands, namely, PO₄³⁻ν3 modes for asymmetric HA (1030˜1033 cm⁻¹ and 1097˜1110 cm⁻¹) and PO₄³⁻ν4 modes (between 601-607 and 563-569 cm⁻¹). The intensities of these bands were in proportion to the HA amount of the composite membranes. It could be confirmed from these facts that the composite membranes in accordance with the present invention were composites of collagen and HA.

(2) Morphology of the Composite Membrane

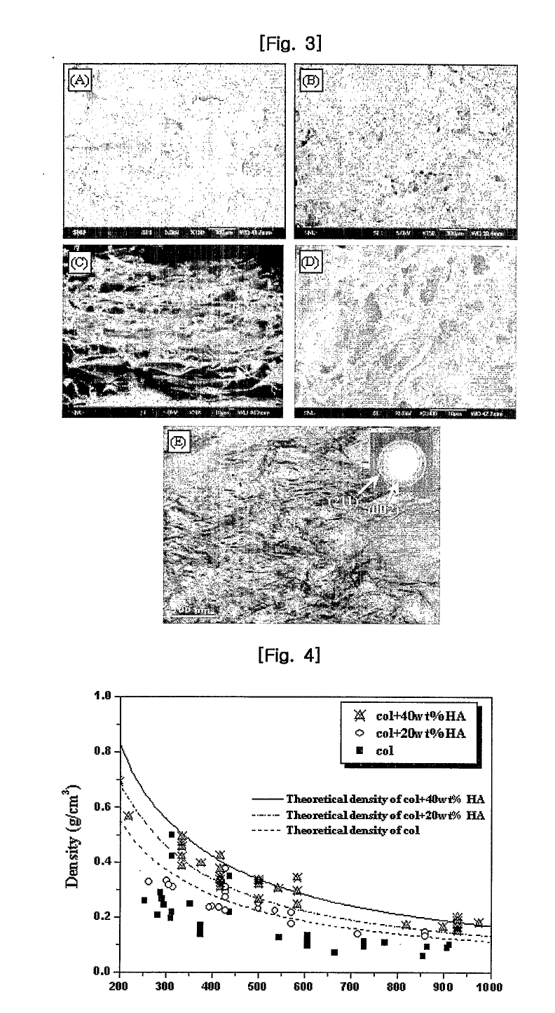

FIG. 3 is microscopic images showing morphologies of the collagen/hydroxyapatite composite membranes produced by the examples according to the present invention, wherein (A) is an SEM image showing a surface of a cross-linked pure collagen membrane, and as SEM images of the cross-linked collagen/hydroxyapatite composite membrane with 40% by weight (nominal component ratio) of hydroxyapatite, (B) is for a surface, (C) is for a cross-section, and (D) is for exposed microfibers, and (E) shows a TEM image and SAD pattern.

As shown in FIGS. 3(A) and (B), the surface of the collagen/hydroxyapatite composite membrane with the nominal composition of 60/40 was almost similar to the surface of the pure collagen membrane, but had little rougher and smaller pores than those of pure collagen membrane. On the other hand, a very little amount of HA nano-scale crystals was formed on the surface of the collagen/hydroxyapatite composite membrane with the nominal composition of 80/20 (not shown in Figs.), but HA nano-scale crystals were almost formed on that of the collagen/hydroxyapatite composite membrane with the nominal composition of 60/40. As shown in FIG. 3(C), the cross-section of the composite membrane had a lamellar structure with porous layers between the surfaces. This structure was commonly formed at the composite membrane prepared by filtering method, regardless of the amount of hydroxyapatite. FIG. 3(D) showed a part of the composite membrane magnified 2000 times in which the collagen fibers were partly protruded and hydroxyapatite covered over the surface of the membrane. It was shown on FIG. 3(E) that hydroxyapatite was distributed in a form of needle phase with several hundred nano size and it was confirmed from the SAD image that hydroxyapatite was arranged along with the 002 direction. It was therefore confirmed that the axis of hydroxyapatite was consistent with the direction of collagen fiber.

The composite membranes in accordance with the examples of the present invention have entirely dense surfaces with slight pores and porous cross-sectional structure with porous layers. Such dual structure allows the dense surface layer to prevent the infiltration of connective fibers into defective sites and the porous cross-sectional structure to induce differentiation of osteoblasts and to fill the connection of bones with the membrane in the pores of membrane.

(3) Mechanical Properties

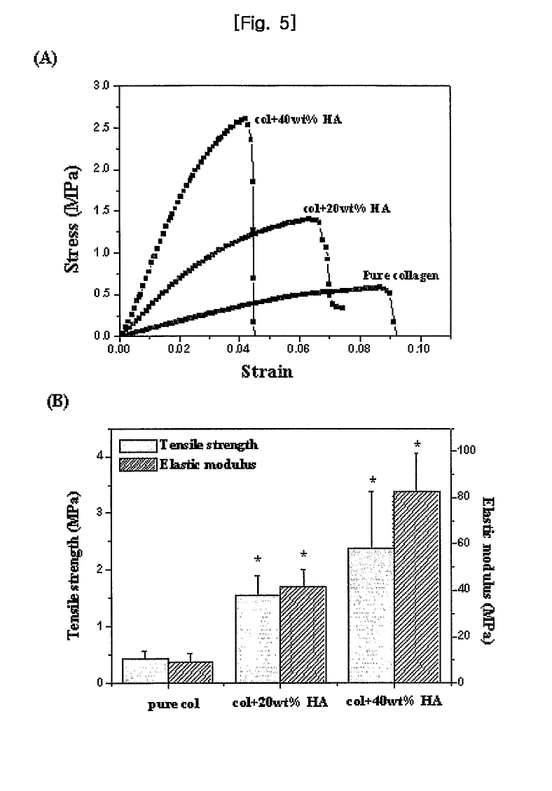

FIG. 4 is a graph showing density distributions of the cross-linked collagen/hydroxyapatite composite membranes produced by the examples according to the present invention with thicknesses, wherein all the specimens have same areas by filtering with same means and method. FIG. 5 is a graph showing the mechanical properties of the cross-linked collagen/hydroxyapatite composite membranes produced by the examples according to the present invention, wherein (A) is a curve of stress-strain, and (B) shows tensile strength and elastic modulus (n>6, *p<0.01).

The solid and dotted lines in FIG. 4 are theoretical densities which were calculated on the assumption that the amounts of collagens were same in all the membranes and the amounts of HA resulted from TGA. The membranes were denser from the result of FIG. 4 when the thickness became thinner. This was natural result because the membranes were formed with the same area by filtering method. The amounts of reactants and precipitations were predetermined. As such, the membranes of the present invention can have suitable densities by adjusting the thickness in the filtering and subsequent pressing processes. The yield of HA formation and precipitation can be recognized from the difference between the actual and theoretical densities. Density can largely affect the porosity and property of membrane, and the present invention can allow membranes to be made suitable for certain uses by controlling the thickness.

The results of FIG. 5 were those of testing the dumbbell-shaped specimens of the membranes with the thickness of 500-600□ which were considered to be porous in the range. As shown in FIG. 5, when the amount of hydroxyapatite became larger, the membrane became more brittle and had more tensile strength. The elastic modulus also increased with the increase of hydroxyapatite amount. In order to be used as GBR membrane, the membrane has to be appropriately high in tensile strength to the extent the membrane has no sinking or destruction in body. The composition of hydroxyapatite will make a positive effect in such viewpoint. FIG. 5 showed that the composite membranes of the present invention were excellent in mechanical properties in comparison with the pure collagen membrane.

(4) Form Stability

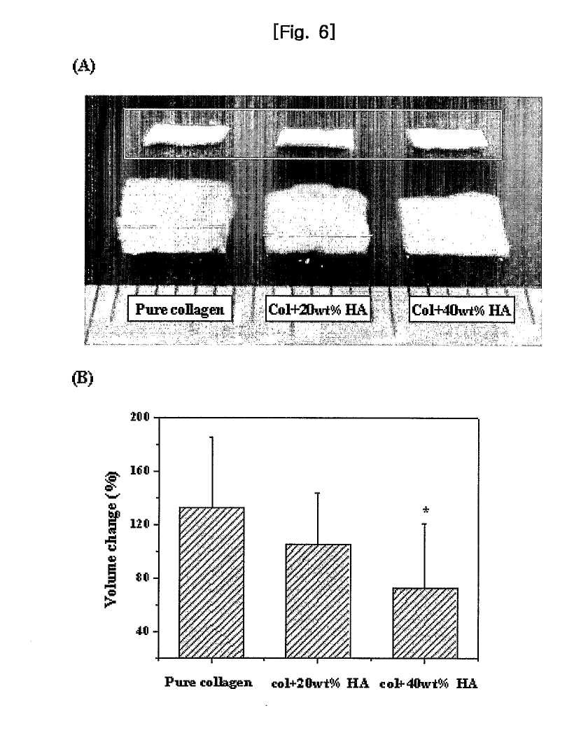

FIG. 6 shows form stabilities of the cross-linked collagen/hydroxyapatite composite membranes produced by the examples according to the present invention, wherein (A) is a photograph showing the level of swelling in water and (B) is a graph showing the change of volume after the membranes according to the present invention were immersed into water for 24 hours (*p<0.05).

Since the main environment in the human body is consisted of water, the behavior of the composite membrane in water also is an important factor. The one example of such behavior is volume change. It is reported that the natural polymers generally swell in water and therefore has high volume change. However the small volume change is better for the use of GTR. It is because the form may be easily destroyed when the volume change is high. In this term, hydroxyapatite positively affects the formability of the composite membrane. It could be recognized from FIG. 6 that the volume change of membrane was smaller when the amount of hydroxyapatite was higher in the composite membrane. While the pure collagen membrane was changed to two times or more in water, the volume change of the collagen/hydroxyapatite composite membrane with the nominal composition of 60/40 was considerably less than that of the pure collagen membrane.

The swelling property of the collagen membranes are affected in accordance with the degree of cross-linkage. However, FIG. 6 showed the results for the membranes as considered to have the same degree of cross-linkage. Therefore, it may be considered from the results that the swelling property may be affected by the other factors than the degree of cross-linkage. In general, the mechanical properties such as tensile strength and stiffness can affect space-maintaining capacity or form stability of the membrane. It was reported that collagen/hydroxyapatite composite with 80% by weight of HA could enhance the compressive strength in comparison to the dense pure HA due to the reinforcement of collagen in the composite. On the other hand, the composite membrane of the present invention enhanced the mechanical properties in comparison to the pure collagen membrane, and so, it can be assumed from the results that HA nano-crystals coated on the collagen fiber reinforce the collagen membrane. However, in some cases, the mineralized particulates composed on the membrane may cause cracks on the defective sites of membrane to decrease the tensile strength and increase the elastic modulus. Accordingly, it is difficult to apply the general relationship between the mechanical properties and the form stability to any actual events. But, it can be said that the present invention has achieved the mechanical properties of the composite membrane simultaneously with the form stability.

(5) Biological Stability (Biodegradability)

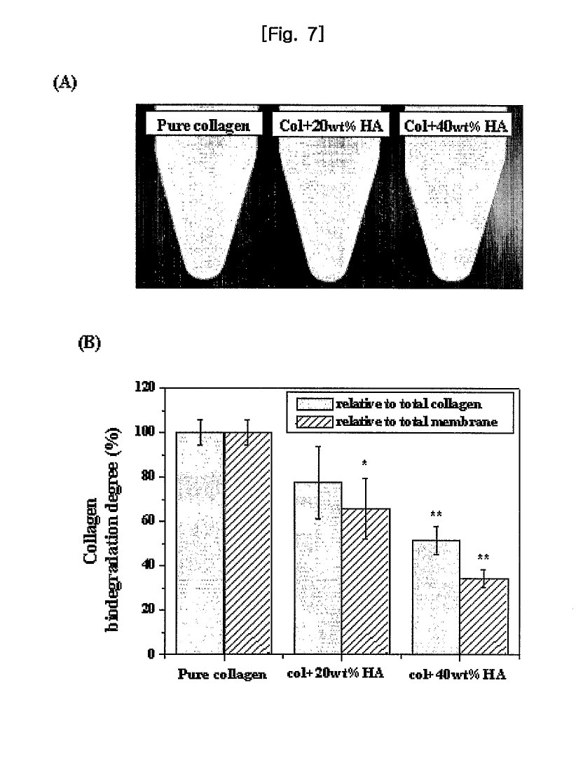

FIG. 7 shows biodegradable stabilities of the cross-linked collagen/hydroxyapatite composite membranes produced by the examples according to the present invention, wherein (A) is a photograph showing the status after the membranes according to the present invention were treated with 150 U/ml of collagenase solution for 24 hours, and (B) is a graph showing the level of collagen biodegradability. All the membranes used in (B) were cross-linked by EDC/NHS and cultivated with 150 U/ml of collagenase at 37° C. for 24 hours. The values are average±standard deviation (*p<0.05, p<0.01).

Collagen can be decomposed by collagenase in a human body. Efforts have been made to increase the biological stability of collagen through the cross-linkage of collagen for preventing fast degradation of collagen. Such degradation rate is an important factor for the biodegradable membrane to be used in the procedures of GTR or GBR. Such degradation rate has to be controlled in consideration of the cure period for the regeneration of the defective bone. Many biodegradable membranes and some commercially available collagen membranes failed to repair the defective bones due to rapid collapse or inflammatory reaction.

The examples of the present invention could enhance the resistance against the collagenase by cross-linkage of collagen and composition of collagen with hydroxyapatite. The membranes used in the examples of the present invention went through the cross-linking reactions with the same levels. As shown in FIG. 7, the pure collagen membrane was completely decomposed after the degradation reaction by the collagenase for 24 hours. The collagen/hydroxyapatite composite membrane with 20% by weight of hydroxyapatite could not maintain the shape of membrane, a part of the membrane being in the form of precipitation, while the collagen/hydroxyapatite composite membrane with 40% by weight of hydroxyapatite could maintain the shape of membrane, about 50% of the collagen remaining without degradation.

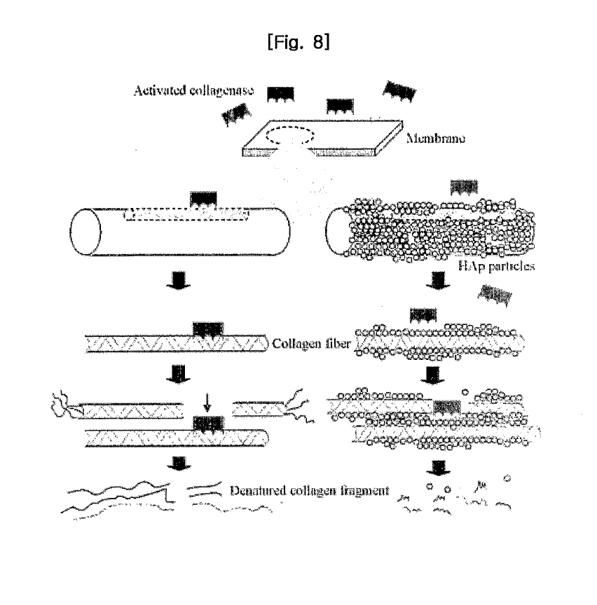

FIG. 8 is a schematic diagram showing a mechanism of collagen biodegradation by collagenase. Collagenase strongly binds and interacts with collagen fiber for degradation of collagen. In theory, about 10% of the collagen molecules in the collagen fibers are exposed for the bind with the enzyme. However, hydroxyapatite on the collagen fibers in the composite membranes in accordance with the examples of the present invention could prevent the enzyme from binding with the collagen to enhance the resistance against the biodegradation by collagenase. Such resistance against biodegradation can be more highly achieved by the uniform precipitation of hydroxyapatite on the collagen fibers.

(6) Biocompatibility (Cellular Activity)

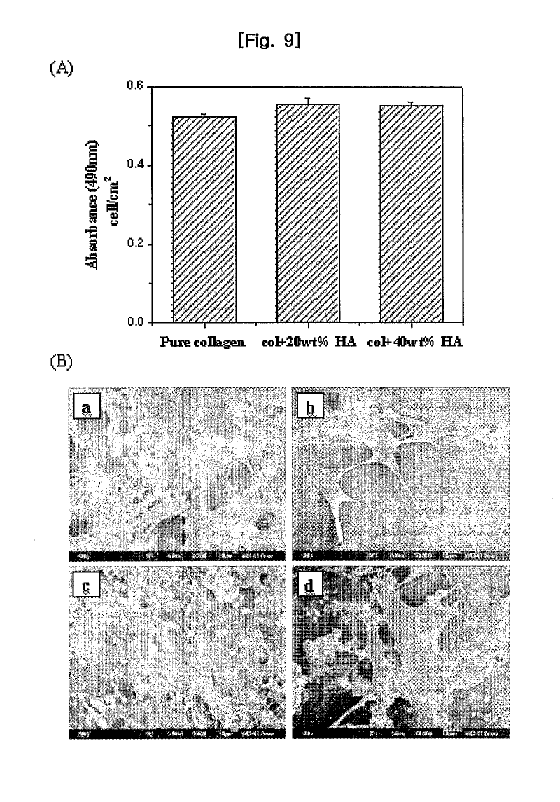

FIG. 9 shows cell characteristics of the cross-linked collagen/hydroxyapatite composite membranes produced by the examples according to the present invention, wherein (A) is a graph showing the characteristics of cell growth and (B) is photographs of SEM images, 3 days later after MT3T3 osteoblasts were cultivated on the membranes. In the (B), (a) and (b) are for pure collagen membrane, and (c) and (d) are for the membrane with 80% by weight (nominal component ratio) of collagen. The amount of cells was not largely different. Granulates were formed on the composite membrane and near the cells on the composite membrane.

As shown in FIG. 9, the osteoblasts spread on the membranes were well grown. It was difficult to say which one among the pure collagen membrane and the composite membranes would better in cellular characteristics because the osteoblasts were well grown on all the membranes. However, mineralized granulates were observed on the composite membranes at 3 days culturing, and which were not on the pure collagen membrane. According to the recent reports [S. Bar, G. Torun Kose, V. Hasirci Bone tissue engineering on patterned collagen films: an in vitro study, Biomaterials 2005; 26: 1977-1986; K. Fujihara, M. Kotaki and S. Ramakrishna, Guided bone regeneration membrane made of polycaprolactone/calcium carbonate composite nano-fibers, Biomaterials 2005: 26; 4139-4147], it could be considered from results of in vitro studies that calcium phosphate in collagen composites hindered the cell proliferation of osteoblast, but induced the differentiation of bone cells, which exhibited a great enhancement of ALP activity in comparison to native collagen. Nacre recognizable as sign of mineralization was observed on the cellular surface of Ca-rich polymer membrane consisting of polycaprolactone and calcium carbonate one day after the cell cultivation. Such mineralization might be considered to be associated with cell differentiation.

It can be expected from the observation and conclusion as explained above that the mineralized granulates occurring on the surface of the composite membrane in accordance with the examples of the present invention are to indicate that the composite membrane of the present invention will positively affect the differentiation of osteoblasts and repair the bone defect successfully.

### INDUSTRIAL APPLICABILITY

The collagen/apatite composite membrane prepared in accordance with the present invention has excellent mechanical properties, biocompatibility and suitable biodegradability and therefore it can sufficiently play a role of the barrier to prevent connective fibers from infiltrating into the defective sites in the procedures of GTR and GBR and can help the differentiation of osteoblasts in the defective sites. Accordingly, the composite membrane of the present invention can be used for GTR and GBR.

1. A method for producing a collagen/apatite composite membrane comprising the steps of:
preparing aqueous solution containing a soluble calcium ion (calcium ion solution);
preparing phosphate/collagen solution in which collagen is dissolved into aqueous solution (phosphate solution) containing soluble phosphate ion;
preparing buffer solution adjusted with pH 7 or higher;
adding said calcium ion solution and said phosphate/collagen solution into said buffer solution and stirring the mixture to form precipitation of collagen/apatite composite; and,
washing and filtering such precipitation of collagen/apatite composite on a filter paper and then lyophilizing the precipitation to obtain a collagen/apatite composite membrane.

2. The method for producing a collagen/apatite composite membrane as claimed in claim 1, wherein the method further comprises the step of cross-linking the collagen of said collagen/apatite composite membrane.

3. The method for producing a collagen/apatite composite membrane as claimed in claim 2, wherein said cross-linking of collagen is performed by immersing said collagen/apatite composite membrane into EDC-NHS solution in alcohol in which 1-ethyl-3-(3-dimethyl aminopropyl)carbodiimide (EDC) and N-hydroxysuccinimide (NHS) is dissolved in alcohol.

4. The method for producing a collagen/apatite composite membrane as claimed in claim 3, wherein said alcohol used as a solvent in said EDC-NHS solution is ethanol.

5. The method for producing a collagen/apatite composite membrane as claimed in claim 2, wherein the method further comprises the steps of, after said cross-linking, recovering and washing said cross-linked membrane with pure water and then lyophilizing it.

6. The method for producing a collagen/apatite composite membrane as claimed in claim 5, wherein the method further comprises the step of adjusting the thickness of said membrane by drying said membrane in the condition of uniformly pressing the surface of said membrane at a wet atmosphere.

7. The method for producing a collagen/apatite composite membrane as claimed in claim 1, wherein said calcium ion solution and said phosphate/collagen solution is prepared to allow the nominal component ratio of collagen and apatite, which could be obtained in the case if the reaction to form said precipitation of collagen/apatite composite were perfectly performed without any loss, to be a value in the range of 8:2˜6:4 by weight.

8. The method for producing a collagen/apatite composite membrane as claimed in claim 1, wherein said calcium ion solution is solution in which Ca(OH)₂ is dissolved into water.

9. The method for producing a collagen/apatite composite membrane as claimed in claim 1, wherein said phosphate/collagen solution is solution in which collagen is dissolved into aqueous H₃PO₄ solution.

10. The method for producing a collagen/apatite composite membrane as claimed in claim 1, wherein said step of forming the precipitation of said collagen/apatite composite is performed on the condition that the mixture is kept at pH 7 or higher.

11. The method for producing a collagen/apatite composite membrane as claimed in claim 1, wherein said buffer solution is tris-HCl buffer solution established at pH 9 and said step of forming the precipitation of said collagen/apatite composite is performed on the condition that the mixture is kept at pH 9.

12. The method for producing a collagen/apatite composite membrane as claimed in claim 1, wherein said steps of washing and filtering the precipitation of said collagen/apatite composite is performed by the steps of separating such precipitation by means for separating solid and liquid, washing the precipitation and then casting the precipitation onto a filter paper, or said steps of washing and filtering is performed by the steps of casting such precipitation onto a filter paper and then washing it on the filter paper.

13. The method for producing a collagen/apatite composite membrane as claimed in claim 1, wherein the apatite in said collagen/apatite composite is hydroxyapatite.

14. A collagen/apatite composite membrane produced by a method as claimed in a claim among claims 1 to 13.
